(19) United States
(12) Plant Patent Application Publication
(10) Pub. No.: US 2015/0257326 P1
(43) Pub. Date: Sep. 10, 2015

(54) Euphorbia milii plant named '12-03-HE'

(50) ;

(71) Applicant: Steen Thomsen, Sonderso (DK)

(72) Inventor: Steen Thomsen, Sonderso (DK)

(73) Assignee: Hjortebjerg Plant License, Sonderso (DK)

(21) Appl. No.: 14/544,909

(22) Filed: Mar. 6, 2015

(30) Foreign Application Priority Data
Mar. 7, 2014 (QZ) 201403295

(51) Int. Cl. A01H 5/00

(52) U.S. Cl. PLT/302

(57) ABSTRACT

A new and distinct cultivar of Euphorbia milii plant named ‘12-03-HE,’ particularly characterized by its medium plant height and width; erect, very compact growth habit; grouped disposition of spines; leaves of medium width with the color of the upper side and under side being dark green; and upper side color of its flowers being RHS 155A with green/yellow edges.

### CROSS REFERENCE TO RELATED APPLICATIONS

This application claims priority under 35 U.S.C. §119(f) of the Community Plant Variety Office (CPVO) application No. 201403295 filed Mar. 7, 2014.

### LATIN NAME OF THE GENUS AND SPECIES OF THE PLANT CLAIMED

Euphorbia milii Desmoul.

### VARIETY DENOMINATION

‘12-03-HE.’

### BACKGROUND OF THE INVENTION

The present invention relates to a new and distinct cultivar of Euphorbia plant, botanically known as Euphorbia milii Desmoul., commonly known by the name of Crown of Thorns, and hereinafter referred to by the name ‘12-03-HE.’

The new Euphorbia cultivar is a product of a planned breeding program conducted by the inventor, Steen Thomsen, in Hjortebjergvej, Denmark. The new Euphorbia cultivar originated from selection from random crossing of proprietary seedlings of Euphorbia milii cultivars. The inventor selected the new Euphorbia milii cultivar as a single flowering plant from the progeny of the above polycrosses on the basis of the color of its flowers and its erect, compact growth habit.

Asexual reproduction of the new cultivar by terminal cuttings taken and propagated in trial production batches in Hjortebjergvej, Denmark, has demonstrated that the unique features of this new Euphorbia cultivar are stable and reproduce true-to-type in many successive generations of asexual reproduction.

### BRIEF SUMMARY OF THE INVENTION

The following traits have been repeatedly observed and are determined to be the unique characteristics of ‘12-03-HE.’ These characteristics in combination distinguish ‘12-03-HE’ as a new and distinct Euphorbia cultivar:

1. Plant height of about 16-18 cm and plant width of about 15-17 cm.

2. Grouped disposition of spines.

3. Leaf width about 2 cm.

4. Leaf color on the upper and under sides being dark green.

5. The color of the upper side of its flowers is RHS 155A with green/yellow edges.

6. Erect, very compact growth habit.

Plants of the cultivar ‘12-03-HE’ can be compared to plants of the Euphorbia milii cultivar ‘DINNI’. In side-by-side comparisons conducted by the inventor in Denmark, plants of the new cultivar ‘12-03-HE’ and the comparison cultivar ‘DINNI’ differ in the following characteristics:

1. Plants of ‘12-03-HE’ have erect, very compact growth habit, whereas plants of ‘DINNI’ have erect/semi-erect, broad growth habit.
2. Plants of ‘12-03-HE’ have flowers with upper side color being RHS 155A with green/yellow edges, whereas plants of ‘DINNI’ have flowers with upper side color being RHS 010C with rose edges.

### BRIEF DESCRIPTION OF THE DRAWINGS

The accompanying color photographs illustrate the overall appearance and details of flower form, color and structures of the new cultivar ‘12-03-HE’, showing the colors as true as it is reasonably possible to obtain in color reproductions of this type. Colors in the photographs may differ slightly from the color values cited in the detailed botanical description, which more accurately describe the actual colors of the new Euphorbia cultivar.

FIG. 1 shows a close up of typical flowers of ‘12-03-HE.’

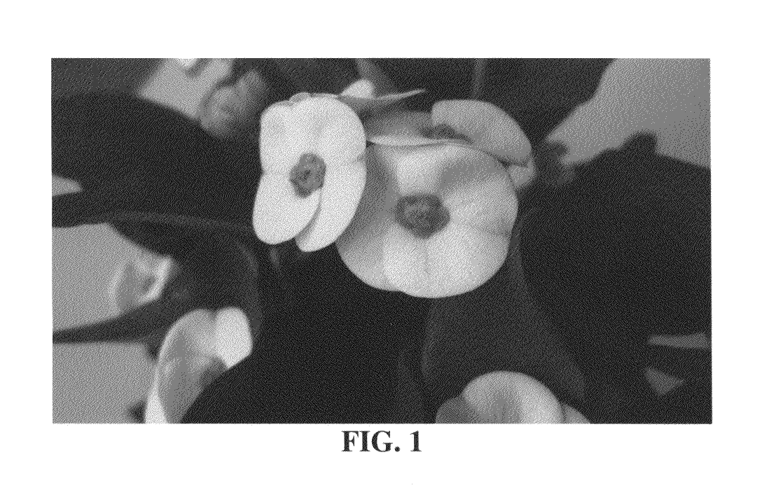

FIG. 2 shows a side view of a typical flowering plant of ‘12-03-HE’ as grown in an 11 cm pot.

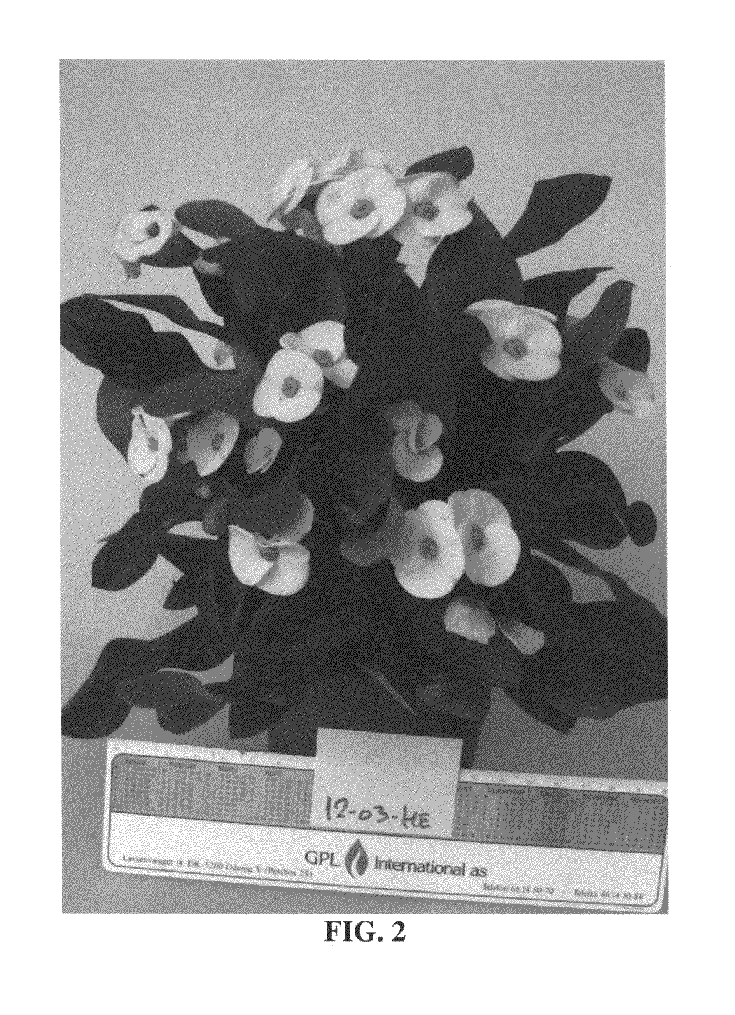

FIG. 3 shows a top view of a typical flowering plant of ‘12-03-HE’ as grown in an 11 cm pot.

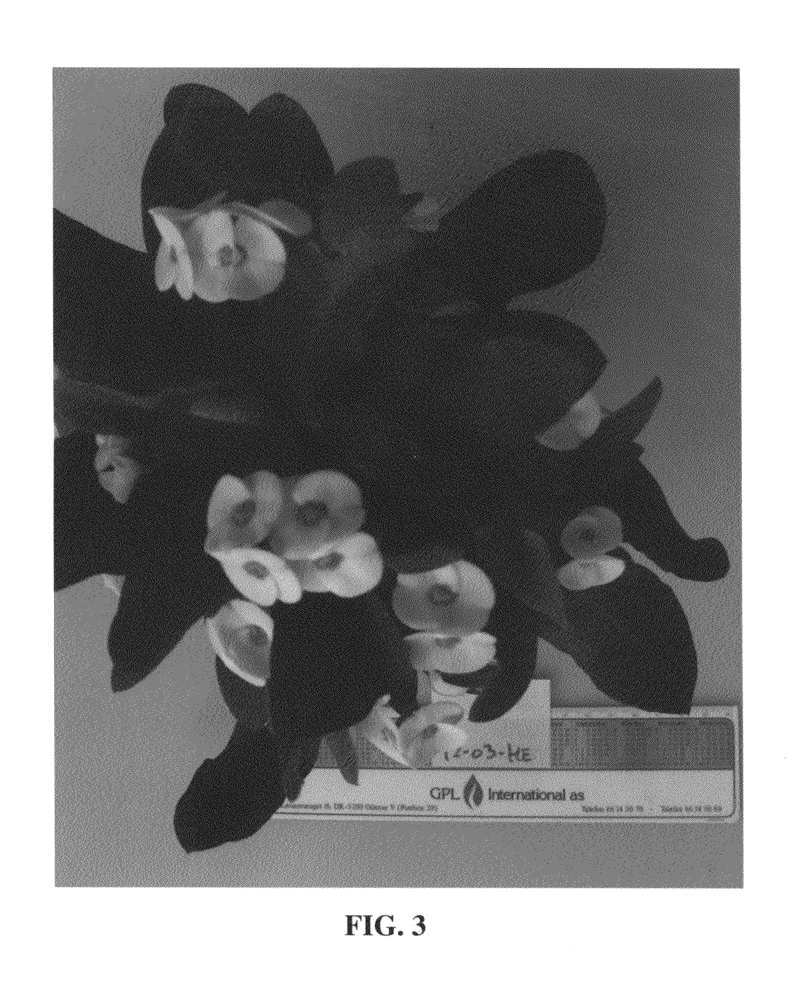

### DETAILED BOTANICAL DESCRIPTION

Plants of the new cultivar ‘12-03-HE’ have not been observed under all possible environmental conditions. The phenotype may vary somewhat with variations in environment such as temperature, light intensity, day length, and fertility level without, however, any variance in genotype.

The aforementioned photographs, together with the following observations, measurements and values describe plants of the new cultivar ‘12-03-HE’ as grown in Hjortebjergvej, Denmark, under conditions which closely approximate those generally used in commercial practice. Plants of ‘12-03-HE’ were grown under the temperature range of about 20-22° C. for a day length of about 12 hours. Artificial light of intensity of 50 Watt/m² was used when the intensity of the day light was less than 40 Watt/m². Water with an electrical conductivity of about 1.6 ds/m (deciSiemens/meter) was used for irrigation. All plants were grown in 11 cm pot in a glass greenhouse.

Color observations were taken under natural light, and the color references are made to the R.H.S. Color Chart of The Royal Horticulture Society of London, except where general color terms of ordinary significance are used.
Botanical classification: Euphorbia milii Desmoul.
Parentage:
   Female parent.—Unnamed seedling plant of Euphorbia milii.
   Male parent.—Unnamed seedling plant of Euphorbia milii.
Propagation:
   Type cutting.—Terminal vegetative cuttings taken from plants.
   Time to initiate roots.—about 3 weeks.
   Root description.—fibrous.
   Root color.—white.
Plant description:
   Form.—very compact.
   Crop time.—about 15 weeks.
   Vigor.—medium.
   Plant size.—about 16-18 cm from soil level to top of plant plane.
   Foliage description.—leaves alternate, single, lancet shaped, about 4-5 cm in length and about 1.5-2 cm in width, upper and under sides color being dark green.
Flower description:
   Flower arrangement.—on separate flower stalks, flower has no sepals or petals.
   Natural flowering season.—March-September.
   Flower longevity on the plant.—about 3-4 weeks.
   Quantity of flowers and buds per plant.—about 20-30.
   Rate of flower opening.—4-5 per week.
   Fragrance.—none.
   Infloresence size.—spread across plant.
   Flowers.—round shape, upright aspect, diameter about 0.5 cm, height about 0.5 cm.
   Bracts.—two, opposite, partly overlapping at base, upper side color is RHS 155A.
   Glands.—5 glands.
   Peduncles.—variable length, sticky.
   Pedicel.—variable length, sticky.
Reproductive organs:
   Pollen.—brown colored.
   Gynoecium.—red-yellow color.
   Seeds/fruit.—3 seeds per fruit.
   Pest/disease tolerance/susceptibility.—no specific resistance or susceptibility was observed during cultivation, except some resistance against trips.

What is claimed is:

1. A new and distinct cultivar of Euphorbia milii plant named ‘12-03-HE,’ as illustrated and described herein.
